(19) United States
(12) Patent Application Publication
(10) Pub. No.: US 2022/0372464 A1
(43) Pub. Date: Nov. 24, 2022

(54) SYSTEM AND METHOD FOR AUTOMATIC NUCLEIC ACID EXTRACTION AND QUIALITATIVE ANALYSIS

(71) Applicant: TAIWAN ADVANCED NANOTECH INC., Taoyuan City (TW)

(72) Inventors: Chien-Hsing CHIEN, Taoyuan City (TW); Chien-Ju LIN, Taoyuan City (TW); Yi-Hsueh LEE, Taoyuan City (TW)

(73) Assignee: TAIWAN ADVANCED NANOTECH INC., Taoyuan City (TW)

(21) Appl. No.: 17/731,567

(22) Filed: Apr. 28, 2022

Related U.S. Application Data
(60) Provisional application No. 63/190,393, filed on May 19, 2021.

(51) Int. Cl. C12N 15/10

(52) U.S. Cl. CPC C12N 15/1013

(57) ABSTRACT

The present invention provides a system and method for automatic nucleic acid extraction and qualitative analysis. The system comprises a magnetic rotary mixer which comprises a plurality of magnetic rods for generating magnetism, configured to be retractable from the magnetic rotary mixer; a plurality of spin shaft for mounting tips, and the plurality of magnetic rods extend therein; an auto stage comprises a plate holder, which allows a plate place thereon; a mixer holder to hold the magnetic rotary mixer over the plate holder; and a heat plate, disposed under the plate holder for heating the plate. The present invention provides an automated high-throughput nucleic acid extraction and qualitative diagnosis with high efficiency and high accuracy, which is easy to interpret for operators, and realize that nucleic acid extraction and molecular detection can be completed at one time in a single device.

### CROSS-REFERENCE TO RELATED APPLICATION(S)

The application incorporates U.S. Provisional Application No. 63/190,393, filed on May 19, 2021, entitled “SINGLE SYSTEM AND STEP TO ACHIEVE AUTOMATED HIGH-THROUGHPUT NUCLEIC ACID EXTRACTION AND QUALITATIVE METHOD” as reference herein in its entirety.

### FIELD OF THE INVENTION

This research and development result is an automated nucleic acid extraction and qualitative diagnosis. This technology can be used in the field of academic research, clinical pathogen detection, entry-exit inspection operations, and other nucleic acid analysis-related applications. The characteristics of automation and the high-throughput process of this technology are suitable for understaffed units. The simple interpretation method reduces the professional threshold required by operators. In addition, the integrated kit and extraction system realize a single device that can complete nucleic acid extraction and molecular detection at one time.

### BACKGROUND OF THE INVENTION

Since the outbreak of coronavirus disease 2019 (Covid-19), the demand and value of nucleic acid-based pathogen detection technology has increased significantly. Currently, the most common and reliable method to detect the RNA of severe acute respiratory syndrome coronavirus 2 (SARS-CoV-2) is real-time quantitative polymerase chain reaction (qPCR). However, the procedure of qPCR takes a couple of hours and requires well-trained laboratory technicians with expensive equipment. In 2000, Notomi and others further developed the LAMP (loop-mediated isothermal amplification) technology in Japan. Loop-mediated isothermal amplification (LAMP) is an alternate nucleic acid-based detection method that can isothermally produce amplified PCR products resulting in a qualitative diagnosis. This method allows the nucleic acid amplification reaction to be performed at a single temperature (60-65° C.), and the product can yield a thousand times than the traditional PCR. The sensitivity of LAMP can reach a level below 10 copies. In addition, the results of the reaction can be observed through precipitation, fluorescence or color changed, so it is a very convenient and rapid method for nucleic acid testing.

A precise molecular biological testing relies on high-quality and high-efficiency nucleic acid extraction pre-processing. In 2014, the applicant invented the rotating-stirring nucleic acid extraction technology, which automatically extracts the nucleic acid through the magnetic attraction of magnetic bead. The operation time is relatively short and has a low risk of cross-contamination. The extracted nucleic acid can be applied for downstream nucleic acid analysis with real-time PCR (Q-PCR) instrument.

To save the time of extracting nucleic acid, amplifying nucleic acid and analysis of nucleic acid, and further improving the purity of the nucleic acid, combining the extraction and analysis into a one-time automatic process may help. Therefore, a single system and step to achieve automated high-throughput nucleic acid extraction and qualitative method should be needed.

### SUMMARY OF THE INVENTION

For the purpose of the present disclosure, providing a system for automatic nucleic acid extraction and qualitative analysis, comprising: a magnetic rotary mixer, comprises: a plurality of magnetic rods for generating magnetism, configured to be retractable from the magnetic rotary mixer; a plurality of spin shaft for mounting tips, and the plurality of magnetic rods extend therein; an auto stage, comprises: a plate holder, which allows a plate place thereon; a mixer holder to hold the magnetic rotary mixer over the plate holder; and a heat plate, disposed under the plate holder for heating the plate.

Preferably, the plate holder is horizontally movable.

Preferably, the plate holder is moved by a stepper motor.

Preferably, the mixer holder is vertically movable.

Preferably, the mixer holder is moved by a stepper motor.

Preferably, the magnetic rotary mixer comprises 8 spin shafts.

Preferably, the magnetic rotary mixer further comprises a control panel for controlling a condition of the nucleic acid extraction.

Preferably, the plate has 96 wells.

Preferably, the system further comprises a cover shell.

Preferably, the spin shaft is rotated by a motor.

Preferably, the auto stage comprises a controlled chip with preset programs.

For another purpose of the present disclosure, providing a method for automatic nucleic acid extraction and analysis performed by the above system, comprising: introducing samples, reagents and beads into the plate; conducting a nucleic acid extracting step, the magnetic rotary mixer mixes the samples, the reagents and the beads, and extracts the nucleic acid thereof with the beads; and conducting an analysis step by RT-LAMP, wherein the plate and the magnetic rotary mixer are moved automatically when conducting the nucleic acid extracting step.

Preferably, the plate and the magnetic rotary mixer are moved by the stepper motor.

Preferably, the plate and the magnetic rotary mixer are moved horizontally and vertically respectively.

Preferably, the method further comprises a heating step for controlling the temperature of assay step.

Preferably, the heating step is performed by the heat plate.

Preferably, a reagent of RT-LAMP comprises primer that can combine with the nucleic acid and moderate pH.

Preferably, the reagent of RT-LAMP further comprises pH indicator.

Preferably, the beads are magnetic beads

The automated system disclosed in the present disclosure is designed for mid- to-high throughput nucleic acid extraction application. Specialized spin tips bring in high efficiency in mixing samples, the isolation principle is the collection and transfer of magnetic beads which adsorbs nucleic acid from well to well, and purified DNA and RNA can be obtained after binding, wash, and elution. As such, through using the system for automatic nucleic acid extraction and qualitative analysis disclosed in the present disclosure, user may save more time and labor to obtain a high efficiency nucleic acid extraction and analysis application.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1A illustrate a perspective view of an automatic nucleic acid extraction and qualitative analysis system of an embodiment of the present disclosure, and FIG. 1B illustrates the system further comprises a cover shell.

FIG. 2 illustrates a magnetic rotary mixer of an embodiment of the present disclosure.

FIG. 3 illustrates different states of a magnetic rotary mixer of an embodiment of the present disclosure. (A) of FIG. 3 illustrates the magnetic rods extending from the magnetic rotary mixer through the spin shaft, (B) of FIG. 3 illustrates the magnetic rods retracted into the magnetic rotary mixer.

FIGS. 4 illustrates the beads being collected by the magnetic rod of a magnetic rotary mixer of the automatic nucleic acid extraction and qualitative analysis system of an embodiment of the present disclosure.

FIGS. 5 illustrates the beads being released by the magnetic rod of a magnetic rotary mixer of the automatic nucleic acid extraction and qualitative analysis system of an embodiment of the present disclosure.

FIG. 6 illustrates the perspective view of an auto stage of an embodiment of the present disclosure.

FIGS. 7A to 7D illustrate different states while performing a nucleic acid extraction of the automatic nucleic acid extraction and qualitative analysis system of an embodiment of the present disclosure.

FIG. 8 illustrates a schematic drawings of RNA extraction and LAMP detection principle of an embodiment of the present disclosure.

FIG. 9 illustrates the result of RT-LAMP with pH indicator of an embodiment of the present disclosure.

FIG. 10 illustrates the result of colorimetric RT-LAMP for detecting SARS-CoV-2 gene of an embodiment of the present disclosure.

FIG. 11 illustrates performance of the automatic extraction and assay system of an embodiment of the present disclosure.

FIG. 12 illustrates cross-contamination test of the automatic extraction and detection system of an embodiment of the present disclosure.

### DETAILED DESCRIPTION OF THE EMBODIMENTS

The present disclosure is depicted by the accompanying drawings, illustrated by embodiments, and described below. The embodiments are implemented in different forms which, however, are not necessarily required to implement or apply the present disclosure. Thus, the different forms of implementation must not be interpreted in a way to limit the embodiments. Features of specific embodiments, steps of a method for constructing and operating specific embodiments, and the sequence of the steps of the method are disclosed hereunder. However, it is also feasible to use any other specific embodiments to achieve identical or equivalent functions and step sequence. Conversely, the embodiments are provided, such that the description hereunder can be thoroughly and completely presented to sufficiently inform persons skilled in the art of the spirit of the present disclosure. Similar reference numerals used in the accompanying drawings denote similar components. Conventional functions or structures are omitted from the description below for the sake of brevity.

Unless otherwise defined, all the technical terms and jargons used hereunder shall have the same meanings as normally understood by persons skilled in the art. If there is any inconsistency between this specification and the comprehension of persons skilled in the art, the definitions-containing specification shall prevail.

Each singular noun used hereunder includes the plural form of the noun without contradicting the context. Each plural noun used hereunder includes the singular form of the noun without contradicting the context. Furthermore, the expression “at least one” and the expression “one or more” used hereunder have the same meaning, and both include one, two, three or more.

The expression “consisting essentially of” used hereunder is for use in defining a composition, method or device, including any materials, steps, features, constituents or components other than what are expressly stipulated. Its restrictive criterion is: the additional materials, steps, features, constituents or components do not significantly affect essential and novel features of an invention claimed. The scope of the expression “consisting essentially of” lies between that of the expression “comprising” and that of the expression “consisting of”.

The relatively broad scope of the present disclosure is defined by numerical ranges and parameters which are approximate for general description. Furthermore, the numerical ranges and parameters inevitably come with standard deviations associated with any examination methods. The aforesaid “about” means that an actual value can be 10%, 5%, 1% or 0.5% greater than or less than a specific value or a limit of a range. Alternatively, the aforesaid “about” means that an actual value falls within an acceptable standard deviation of its mean, depending on the considerations which persons skilled in the art take into account. In addition to the embodiments of the present disclosure, or unless otherwise expressly specified, all the ranges, numbers, numerical values, and percentages (for example, descriptive of the amount of a material in use, a period of time, temperature, operation conditions, numeric proportions, and the like) stated hereunder are each followed by the adverb “about”. Therefore, unless otherwise conversely specified, all the numerical ranges and parameters disclosed hereunder are presented in the form of approximate numerical values and are subject to changes as needed. The numerical values and parameters must be at least interpreted to be applicable to significant figures and general decimal notation. The ranges of the numeral values are each defined with an endpoint and another endpoint or defined as a range between two endpoints. Unless otherwise specified, the ranges of the numeral values disclosed hereunder include their respective endpoints.

There are various nucleic acid extraction methods on the market. However, no matter it is manual or automated extraction methods, additional operating procedures are required if the further nucleic acid analysis is to be performed. At present, the most common way to analyze nucleic acid is the Q-PCR system. The process of Q-PCR includes plate preparation (manual or automatic sampler), sample loading, program setting, analysis progress, and result interpretation. It takes about 2 to 4 hours and the process may increase the possibility of error and contamination. Besides, reagent loading depends on the skill of the operator, the automatic sampler requires additional space, and interpretation of the results requires a trained professional.

To solve the problems mentioned above and achieve the same accuracy (a simpler and rapid nucleic acid analysis), the present disclosure uses an automated nucleic acid extraction instrument developed by the applicant, and a nucleic acid extraction kit containing LAMP reagents to realize one-time automation nucleic acid extraction and analysis technology.

The significant differences between this technology and the existing nucleic acid extraction and analysis methods are:

a. Combine the extraction and analysis into a one-time automatic process.

b. The interpretation is simple and can be observed directly with the naked eye, which does not require extra equipment.

c. The overall operation time is shorter than the real-time PCR system and the sensitivity remains accurate.

Herein after, an automatic nucleic acid extraction and qualitative analysis system 1 of an embodiment of the present disclosure will be describe in detail corresponding with the drawings.

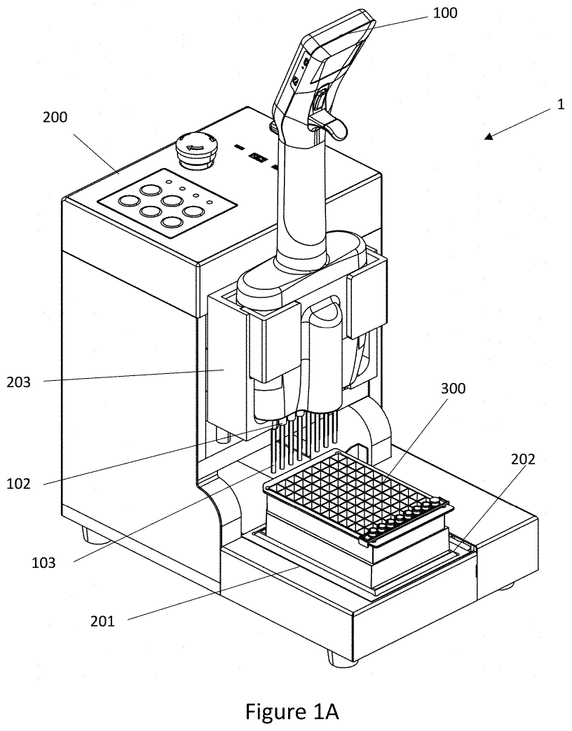

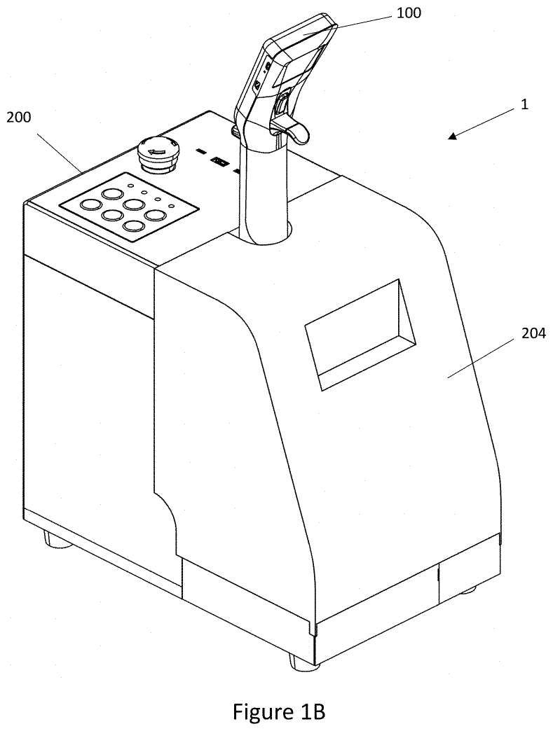

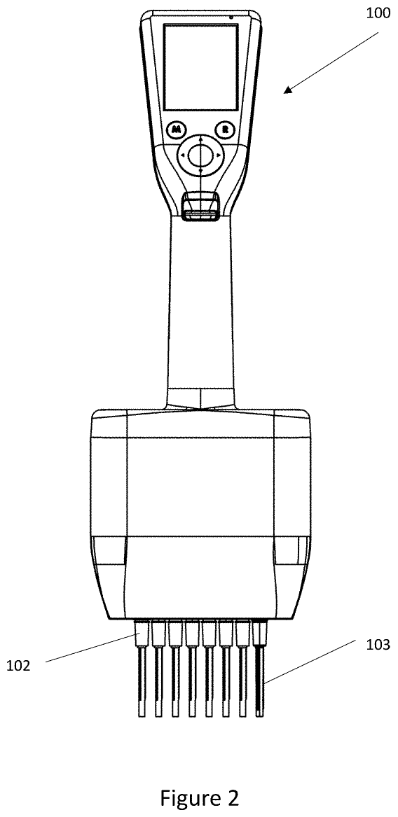

See FIGS. 1A, 1B and 2, FIG. 1A illustrates a perspective view of an automatic nucleic acid extraction and qualitative analysis system 1 of an embodiment of the present disclosure; FIG. 1B illustrates the system 1 further comprises a cover shell 204, and FIG. 2 illustrates a perspective view of a magnetic rotary mixer of an embodiment of the present disclosure.

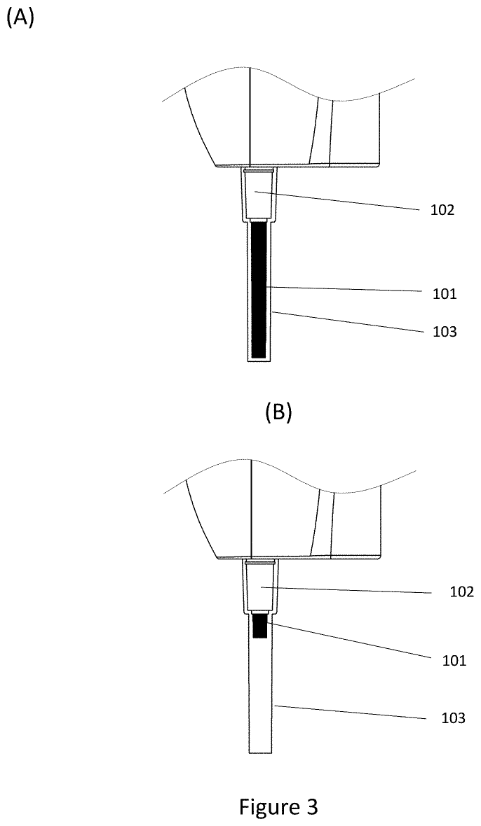

In one embodiment of the present disclosure, please refer to the FIG. 1, the system 1 comprises a magnetic rotary mixer 100, an auto stage 200, and plate 300. The magnetic rotary mixer 100 may comprises a plurality of magnetic rods 101 (see FIG. 3) for generating magnetism, configured to be retractable from the magnetic rotary mixer 100, and plurality of spin shaft 102 for mounting spin tips 103, and the plurality of magnetic rods 101 extend therein. The auto stage 200 may comprises a plate holder 201, which allows the plate 300 place thereon; a mixer holder 203 to hold the magnetic rotary mixer 100 over the plate holder 201; and a heat plate 202, disposed under the plate holder 201 for heating the plate 300. In another embodiment of the present disclosure, the system 1 may further comprise a cover shell 204 to prevent dust or other pollutants.

The plate holder 201 of the auto stage 200 can be movable horizontally. In the automatic extraction and qualitative analysis of the present disclosure, the user only has to prepare the sample from the subjects and the reagents into the corresponding wells of the plate 300, the process will perform automatically. The details of the process will be described later.

To move the plate holder 201 horizontally, the auto stage 200 may comprise a stepper motor. With the stepper motor, the plate holder 201 may move the plate 300 from a well to the next well within a predetermined distance to proceed the nucleic acid extraction process.

On the other hand, for holding the magnetic rotary mixer 100 over the plate holder 201, the auto stage 200 further comprises a mixer holder 203. The mixer holder 203 may hold the magnetic rotary mixer 100 over the plate 300 on the plate holder 201, and move the magnetic rotary mixer 100 vertically with a stepper motor. As such, the magnetic rotary mixer 100 can be moved upward to allow the plate holder 201 move horizontally, and the magnetic rotary mixer 100 can be moved downward to insert the spin tips 103 into the wells.

As shown in FIG. 2, the magnetic rotary mixer 100 comprises a plurality of spin shaft 102. The spin tips 103 may be mounted to the spin shaft 102 and allow the magnetic rods 101 (not shown) extending therein. Since the leading edge of the spin tips 103 are sealed, the reagent will not enter the spin tip 103 and contact the magnetic rods 101. In other embodiment of the present disclosure, the spin tips 103 can be rotated in the wells by rotating the spin shafts through motor to mix and stir the reagent and sample in the wells evenly.

FIG. 3 illustrates the different states of the magnetic rods 101 of the magnetic rotary mixer 100.

In one embodiment of the present disclosure, as shown in (A) of FIG. 3, the magnetic rod 101 extends from the magnetic rotary mixer 100. Particularly, the magnetic rod 101 extends into the spin tip 103 (not shown) mounted to the spin shaft 102. In (B) of FIG. 3, the magnetic rod 101 may retract into the magnetic rotary mixer 100. The magnetic rod 101 is for collecting and releasing the beads during the nucleic acid extraction process. The beads herein described are mentioned about microbeads that may have functional group on the surface itself, and may combine with a target subject, thereby can extract the target subject from sample. The beads may be made of agarose, silicon or any other suitable material which can be absorbed by the magnetic rods 101.

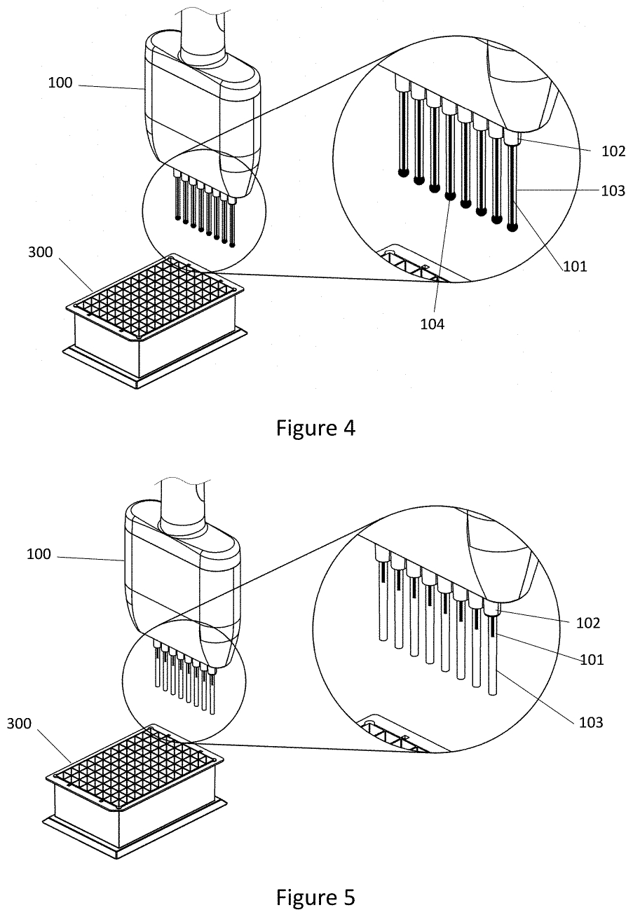

FIGS. 4 and 5 illustrate the beads 104 being collected or released by the magnetic rod 101 of a magnetic rotary mixer of the automatic nucleic acid extraction and qualitative analysis system of an embodiment of the present disclosure respectively.

As shown in FIG. 4, when the magnetic rods 101 extends into the spin tips 103 and provide magnet power, the beads 104 in the wells will be collected around the leading edge of the spin tip 103.

As shown in FIG. 5, when the magnetic rods 101 retract into the magnetic rotary mixer 100, and no longer provides magnet power, so the beads will be released into the wells.

Herein after, the movement of the auto stage 200 will be describe in details.

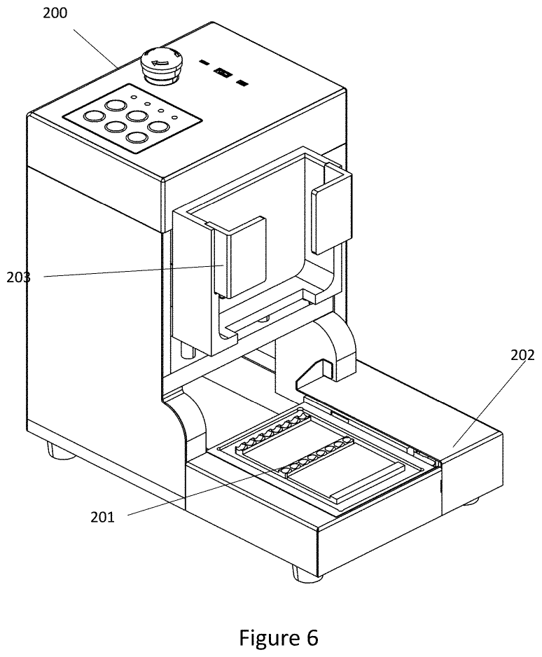

See FIG. 6, auto stage 200 comprises a plate holder 201, a heat plate 202 disposed on the plate holder 201, and a mixer holder 203. The plate holder 201 may hold plate 300 thereon, and comprise a stepper motor (not shown) to perform the horizontal movement of the plate 300. The mixer holder 203 is for holding the magnetic rotary mixer 100 to perform the vertical movement of the magnetic rotary mixer 100. The mixer holder 203 is controlled to be moved by a stepper motor (not shown).

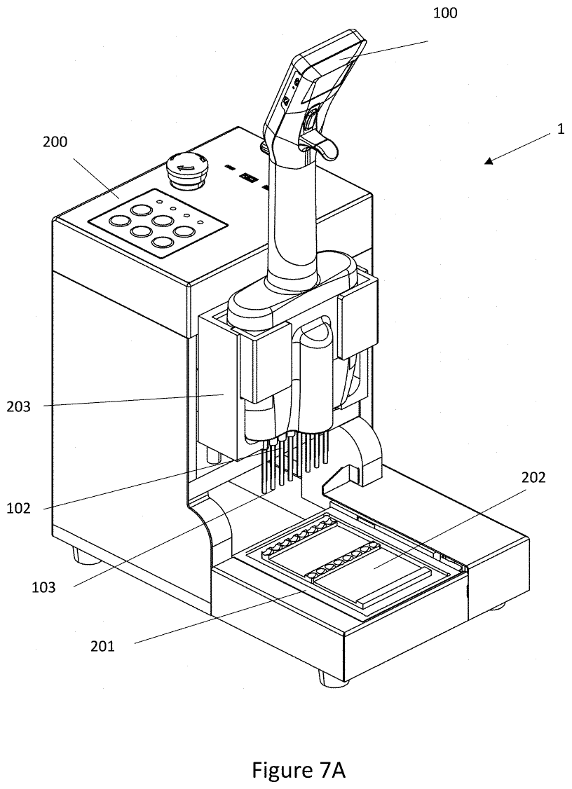

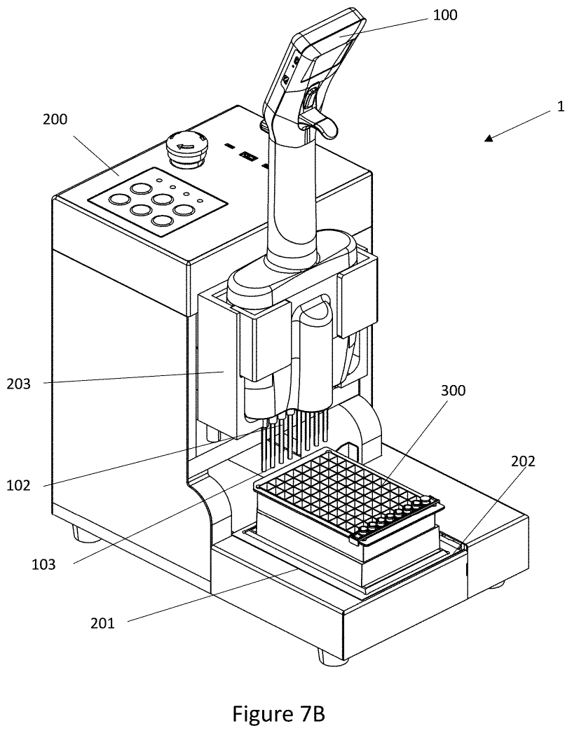

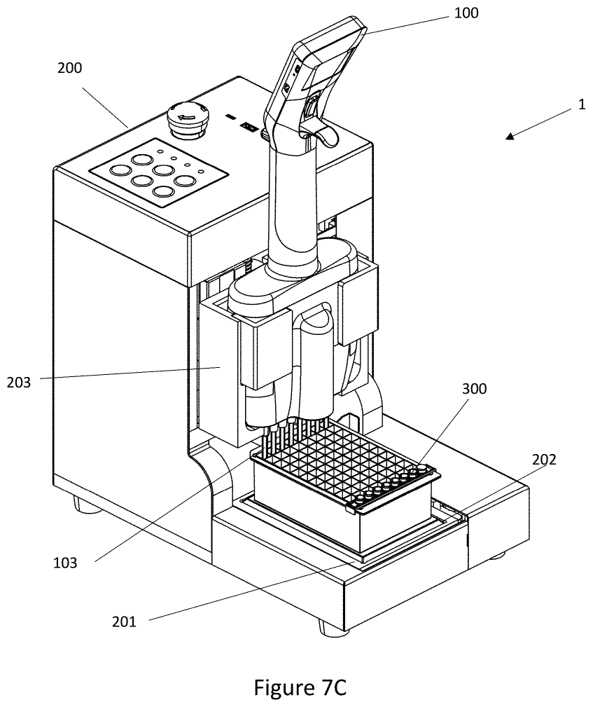

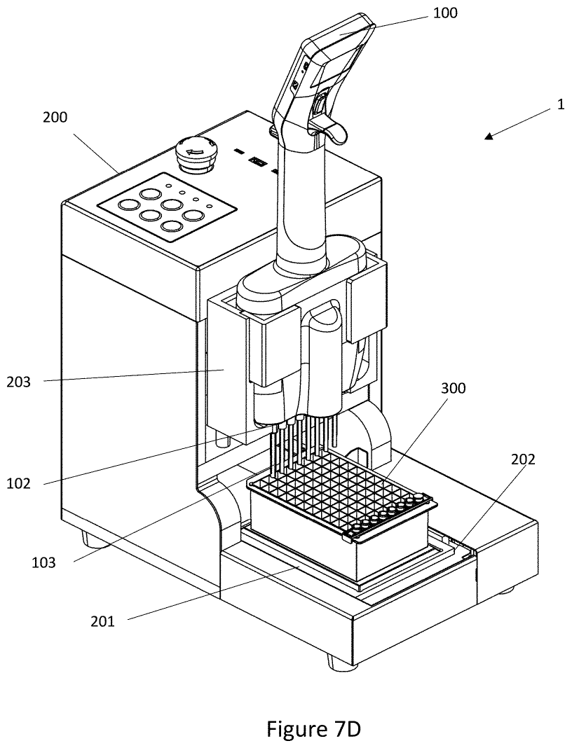

Referring to FIG. 7A to 7D, the operation of the system 1 is shown therein. In FIG. 7A, the mixer holder 203 may hold the magnetic rotary mixer 100 at a preset position before or after the plate 300 to be held on the plate holder 201. The plate holder 202 may move the first row of wells of the plate 300 correspondingly under the magnetic rotary mixer 100 by the stepper motor as shown in FIG. 7B. And then, the stepper motor of the mixer holder 203 may move the magnetic rotary mixer 100 downward, and the spin tips 103 may insert to the first row of wells of the plate 300 as shown in FIG. 7C. Then, rotating the spin shaft 102 to mix the reagent, sample and beads 104 in the first row of wells of the plate 300. Next, the magnetic rods 101 extend from the magnetic rotary mixer 100, and provide magnet power to collect beads 104 around the leading edge of the spin tips 103. The stepper motor of the mixer holder 203 moves the magnetic rotary mixer 100 upward, and the spin tips 103 leave the first row of wells of the plate 300, and the beads 104 are carried by the spin tips 103. And then, the stepper motor of the plater holder 201 moves the plate 300 horizontally to place the second row of the wells under the magnetic rotary mixer 100. The stepper motor of the mixer holder 203 moves the magnetic rotary mixer 100 downward to insert the spin tips 103 to the second row of wells of the plate 300, and the beads 104 can be released in the second row of wells of the plate 300, as shown in FIG. 7D. With the above mentioned operations, the beads 104 may be moved between different rows of wells of the plate 300.

The steps mentioned above may be controlled by a controlled chip. User may set desired steps and programs to the control chip, and the auto stage 200 may be operated automatically.

In a preferred embodiment of the present disclosure, the magnetic rotary mixer 100 may have 8 spin shafts 102 and 8 magnetic rods 101, and the plate 300 may have 96 wells with 8 rows and 12 columns. The present disclosure is not limited thereto, the number of spin shaft, magnetic rods and wells of the plate may be predetermined based on needed.

The method of automatic nucleic acid extraction and assay performed by the above mentioned system 1 comprises following steps: introducing samples and reagents into the plate 300; conducting a nucleic acid extracting step, the magnetic rotary mixer 100 mixes the samples and the reagents, and extracts the nucleic acid thereof with beads 104; and conducting an assay step by RT-LAMP, wherein the plate 300 and the magnetic rotary mixer 100 are moved automatically when conducting the nucleic acid extracting step.

In one embodiment of the present disclosure, the plate 300 and the magnetic rotary mixer 100 are moved by the stepper motor to make sure the movement of the plate 300 and the magnetic rotary mixer 100 are in the correct position. In another embodiment of the present disclosure, the method further comprises a heating step for controlling the temperature of assay step performed by the heat plate 202.

In the assay step by RT-LAMP, the reagent may be introduced after the extraction and amplification of the nucleic acid. And the reagent of RT-LAMP may comprise pH indicator, so that user may recognize the result with their naked eyes.

### EXAMPLE

Herein after, the operation of the system 1 and the method will be described with an example.

### Materials and Methods

### 1. Sample Preparation and Extraction

To test the extraction and detection efficiency of the system in this study, we used synthesized plasmids or commercial pseudovirus (AccuPlex™ SARS-CoV-2 Reference Material, MA, USA) containing the RNA directed against the published CDC and WHO consensus SARS-CoV-2 sequences. The samples were diluted with 1X PBS buffer to proper concentration. Nucleic acids were extracted from 200 μL of samples using a TANBead fully automated magnetic bead operating platform (i.e. the system 1), TANBead Maelstrom™ 8 Autostage (i.e. the auto stage 200) with TANBead Auto Plate (i.e. the plate 300) (Taiwan Advanced Nanotech Inc., Taoyuan City, Taiwan). The auto plate contained 600 μL of lysis buffer, 800 μL of wash buffer, 800 μL of diluted magnetic beads, and 80 μL of elution buffer. The extraction procedure was performed automatically after mounting the tips to magnetic rotary mixer and choosing the program.

### 2. Plasmid Construction

For nucleic acid standards used in our assays, present disclosure synthesized two plasmids on pUC57 vector, which containing the open reading frame of the nucleocapsid protein (N) and envelope (E) protein of SARS-CoV2 respectively. The sequences were based on the Genbank accession number NC_045512.2. The region of N gene plasmid included nucleotides 28,273 to 29,533; the region of E gene plasmid included nucleotides 6,245 to 26,472. The plasmids were transformed into E. coli (DH5α) for amplification and isolated by QIAprep Spin Miniprep Kit (MD, USA). The sequences and maps were attached in supplementary materials.

### 3. Primer Design of RT-LAMP

To design RT-LAMP primer sets for detecting of SARS-CoV-2, present disclosure used NEB LAMP Primer Design Tool (https://lamp.neb.com/#!/). Briefly, using the ORF of N or E gene as input sequence and setting normal default parameters. Present disclosure selected three primer sets from predicted results for following assays, the primer sequences were showed in Table. 1. The primers were then synthesized, and the stocks were dissolved in sterilized ddH₂O in final 100 μM concentration. Six primers (2 μM F3, 2 μM B3, 16 μM FIP, 16 μM BIP, 4 μ M LF and 4 μM LB) were premixed to generate 10X RT-LAMP primer-mix.

TABLE 1
RT-LAMP primer targeting on N and E gene of SARS-CoV-2

| N gene | Primer sequence | E gene | Primer sequence |
|---|---|---|---|
| Set 1 | | Set 1 | |
| E3 | TGGACCCCAAAATCAGCG | F3 | TGAGTACGAACTTATGTACTCAT |
| B3 | GCCTTGTCCTCGAGGGAAT | B3 | TTCAGATTTTTAACACGAGAGT |
| FIP | CCACTGCGTTCTCCATTCTGGTAAATGC | FIP | ACCACGAAAGCAAGAAAAAGAAGTTCGTTTCGGAAGAGACAG |
| | ACCCCGCATTACG | | |
| BIP | CGCGATCAAAACAACGTCGGGCCCTTGC | BIP | TTGCTAGTTACACTAGCCATCCTTAGGTTTTACAACACTCACGT |
| | CATGTTGAGTGAGA | | |
| LF | CCAGTTGAATCTGAGGGTCCACC | LF | CGCTATTAACTATTAACG |
| BP | GGTTTACCCAATAATACTGCGTCTTGG | BP | GCGCTTCGATTGTGTGCGT |
| Set 2 | | Set 2 | |
| F3 | TGGACCCCAAATCAGCG | F3 | TTTCGCAAGAGACAGGTAC |
| B3 | GCCTTGTCCTCGAGGGAAT | B3 | AGGAACTCTAGAAGAATTCAGA |
| FIP | CCACTGCGTTCTCCATTCTGGTAAATGC | FIP | CGCAGTAAGGATGGCTAGTGTAGCGTACTTCTTTTTCTTGCTT |
| | ACCCCGCATTACG | | |
| BIP | CGCGATCAAAACAACGTCGGCCCTTGCC | BIP | TCGATTGTGTGCGTACTGCTGTTTTTAACACGAGAGTAAACGT |
| | ATGTTGAGTGAGA | | |
| LF | GCCAGTTGAATCTGAGGGTCCACC | LF | AGCAAGAATACCACGA |
| BP | ATAATACTGCGTCTTGGTTCACCGC | BP | CGTGAGTCTTGTAAAAC |
| Set 3 | | Set 3 | |
| F3 | AGATCACATTGGCACCCG | F3 | TTTCGGAAGAGACAGGTAC |
| B3 | CCATTGCCAGCCATTCTAGC | B3 | AGGAACTCTAGAAGAATTCAGA |
| FIP | TGCTCCCTTCTGCGTGAGAAGCCAATGC | FIP | CGCAGTAAGGATGGCTAGTGTAGCGTACTTCTTTTTCTTGCTT |
| | TGCAATCGTGCTAC | | |
| BIP | GGCGGCAGTCAAGCCTCTTCCCTACTGC | BIP | TCGATTGTGTGCGTACGTGCTGTTTTTAACACGAGAGTAAACGT |
| | TGCCTGGAGTT | | |
| LF | GGCAATGTTGTTCCTTGAGGAAGTT | LF | ACTAGCAAGAATACCACGA |
| BP | TCCTCATCACGTAGTCGCAACAGTT | BP | CGTGAGTCTTGTAAAAC |

### 4. Colorimetric RT-LAMP Reaction

2X colorimetric buffer contained 2.8 mM dNTP, 20 mM (NH₄)₂SO₄, 16 mM MgSO₄, 100 mM KCl, 0.2% Tween 20, and 200 μM phenol red, the pH value of reaction buffer was adjusted to 8.1 with 1M KOH. RT-LMAP reactions were prepared in a final 25 μ l volume, each reaction mix contained 12.5 μL of 2X colorimetric buffer, 2.5 μLof 10X RT-LAMP primer-mix, 0.07 μL of Bst 2.0 WarmStart DNA Polymerase (New England Biolabs), 0.5 μL of WarmStart RTx Reverse Transcriptase (New England Biolabs), 2 μL of template, and above components were mixed H₂O up to 25 μL. The reactions were incubated at 65° C. for 30 min and observed the color change of the phenol red.

### 5. RT-qPCR of SARS-CoV-2 Gene Detection

To detect genes of SARS-CoV-2 in this study, quantitative PCR was performed with commercial qPCR mastermix, AllplexTM 2019-nCoV assay (Seegene, Inc., Seoul, South Korea). In short, after thawing all reagents completely, PCR setup was prepared by following reagents: 5 μL of 2019-nCoV MOM, 5 μL of 5X realtime one-step buffer, and 5 μL of real-time one-step enzyme. Mixing PCR setup with inverting and spindown, then 8 μL of nucleic acid sample or positive control was added in pre-mix and ready to perform PCR. The RT-PCR assays were performed under the following protocol: reverse transcription at 50° C. for 20 min and initial denaturation at 95° C. for 15 min, 45 cycles of denaturation at 94° C. for 15 s and annealing at 58° C. for 30s using CFX96∜ Real-Time PCR Detection System (Bio-Rad, USA). The results were considered positive if Ct value is less than 40.

### RESULT

### 1. Workflow for the Detection of SARS-CoV-2 using RT-LAMP and Maelstrom 8 Autostage.

To develop a rapid covid-19 diagnosis system, we designed a workflow including sample collection, automated nucleic acid extraction, and RT-LAMP detection. The nasopharyngeal swab specimen is inserted in a sterile tube contained 2 ml of virus transport medium, for storage or following assay. The auto plate is contains lysis buffer, wash buffer, elution buffer, and RT-LAMP reagents.

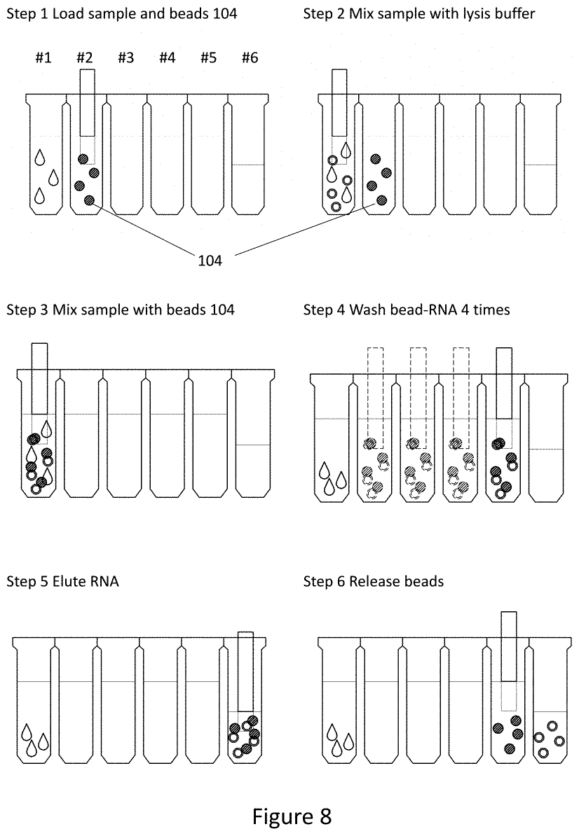

In detail, as shown in FIG. 8, drop shape icon indicates sample, solid circle indicates TANBeads 104 and hollow circle indicates RNA released from sample. Further, the reagents in each well are as described in the below table 2. In step 1, sample is loaded in the first row #1 of the wells, and the TANBeads 104 (i.e. beads 104) is loaded in the second row #2 of the wells with or without washing buffer. The TANBeads 104 may be preloaded in the first row of the wells, too. In step 2, the magnetic rotary mixer 100 mounded with spin tips 103 inserts to the first row of the wells and mix samples with lysis buffer. In step 3, the magnetic rotary mixer 100 moves to the second row of the wells and collects the TANBeads 104. Then, the magnetic rotary mixer 100 with collected TANBeads 104 moves back to the first row of the wells, and mix TANBeads 104 and samples. The designed TANBeads 104 will combine with the RNA lysed by the lysis buffer. In another embodiment, the step 2 may be omitted, the TANBeads 104 may be mixed with samples without the step of mixing samples and lysis buffer. In step 4, the magnetic rotary mixer 100 with TANBeads 104 move to the second row to wash the TANBeads 104. In one embodiment, this step may be performed for 4 times from second row #2 to fifth row #5 of the wells. Then, in step 5, the RNA combined with TANBeads 104 may be released in the sixth row #6 of the wells loaded with RT-LAMP reagents for conducting the following RT-LAMP assay. In step 6, the TANBeads 104 in the sixth row #6 of the wells will be collected by the magnetic rotary mixer 100 and be moved back and released into the fifth row #5 of the wells, and the auto stage 200 incubates the RT-LAMP reagents and the nucleic acids therein at 65° C. for 30 minutes to perform RT-LAMP assay.

In another embodiment, if the user wants to obtain different parts of the nucleic acids for different purposes, for example, one part of nucleic acids for the following RT-LAMP assay and the other parts for other assay, it can be performed by adjusting the times of the elution step. Take two times of the elution step for instance, the fifth row #5 of the wells may be loaded with elution buffer, through controlling the elution time, part of nucleic acids combined with TANBeads 104 may be eluted for other assay, and the rest part of nucleic acids may be bring to the sixth row #6 of the wells for the following RT-LAMP assay as described above.

TABLE 2

| Well | Reagent | Composition |
|---|---|---|
| 1 | Lysis buffer | 45% GuSCN, 10% 15-S-9, |
| | | 45% water |
| 2 | Magnetic Bead | SiO₂ beads |
| 3 | Washing buffer | 40% EtOH, 3% 15-S-9, |
| | | 20% NaCl, 37% water |
| 4 | Washing buffer | 40% EtOH, 3% 15-S-9, |
| | | 20% NaCl, 37% water |
| 5 | Elution buffer | 0.1% Tris, 99.9% water |
| | (or wash buffer) | |
| 6 | Colorimetric detection | 1.4 mM dNTP, 10 mM (NH₄)₂SO₄, |
| | (LAMP mastermix) | 8 mM MgSO₄, 50 mM KCl, 0.1% |
| | | Tween 20, 100 μM phenol red, |
| | | 100 μM mineral oil, 1-unit |
| | | Bst DNA polymerase, and 1-unit |
| | | RTx reverse transcriptase. |
| | | (pH = 8.1) |

Viral genomic RNA will be extracted from the swab specimen using a Maelstrom 8 Autostage in 14 min automatically, which is further dissolved in elution buffer and RT-LAMP reagents. After extraction procedure, auto plate will be incubated at 65° C. for 30 min to perform RT-LAMP reaction. A colorimetric result of RT-LAMP can be observed from the bottom of plate. In addition, the sample dissolved in elution buffer can be used for further analysis, such as real-time PCR. In this study, present disclosure aimed to verify the performance of this extraction and detection system.

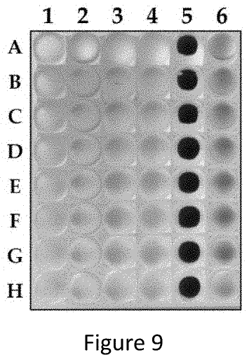

As the RT-LAMP performed, the extracted RNA may be reverse transcript to DNA and amplified, due to pH indicator inside, turning yellow are considered positive and the wells remaining pink are considered negative, see FIG. 9. FIG. 9 illustrates bottom view of auto plate shows the content and colorimetric result. The auto plate is pre-filled with elution buffer, lysis buffer, wash buffer, magnetic beads, and RT-LAMP reagent in order. The target nucleic acid amplified by RT-LAMP resulted in color change of reagent, due to pH indicator inside, turning yellow are considered positive (as shown in position H6); remaining pink are considered negative (as shown in A6 to G6).

### 2. Colorimetric RT-LAMP for Detecting SARS-CoV-2 Gene

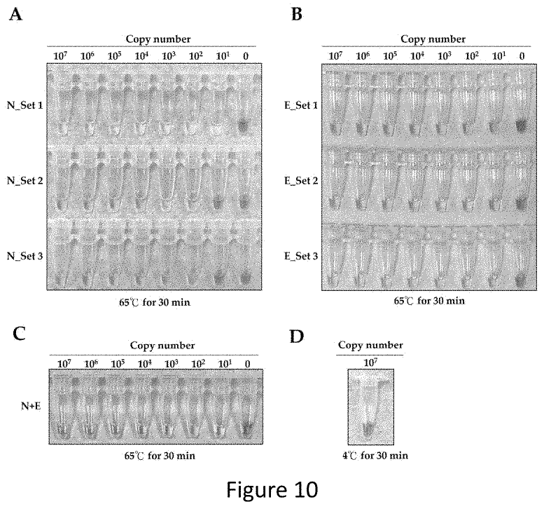

To prepare colorimetric RT-LAMP reagent for covid-19 detection, we synthesized three primer sets or nucleocapsid protein (N) gene and envelope (E) gene of SARS-CoV-2 respectively. The target regions were selected according to the genome reference sequence (NC_045512) on NCBI and the primer sets were generated by NEB LAMP Primer Design Tool. The primer sequences used in this study showed in Table. 1. First, we tested the sensitivities of different primer sets by serial dilution of the standard plasmid. A 10-fold dilution was started from 10⁷ to 10¹ copies per reaction, and the RT-LAMP assays were used 2 μL of the plasmid in a reaction volume of 5 μL. The reactions were assembled on ice and then incubated at 65° C. for 30 min. In N primer set 1 group, 10⁷ to 10¹ copies turned yellow color, and the non-template control remained pink, as observed before reaction started. In N primer set 2 group, 10⁷ to 10² copies turned to yellow color and 10 copies remained pink, as observed in non-template control. Finally, The N primer set 3 group had a similar result with N primer set 2 (FIG. 10, A). Therefore, our data indicated that the N primer set 1 has the best sensitivity and detection limit. On the other hand, 10⁷ to 10¹ copies turned to yellow color in the groups of E primer set 1, 2, and 3. This result suggested that three of E primer sets revealed similar sensitivity (FIG. 10, B).

Next, we tested the detection limit of N/E gene multiplex RT-LAMP by the mix of N and E primer set 1, that based on the results of FIGS. 10, A and B. In FIG. 10, a 10-fold dilution template was added into RT-LAMP reagents with (A) N primer sets,(B) E primer sets, and (C) N+E primer sets, then incubated at 65° C. for 30 min. (D) A 10⁷copies of reaction incubated at 4° C. for 30 min as negative control.

The final concentration of two primer set mix is 4.4 μM (same as individual primer set assay), the ratio of N and E primer set is 1 to 1. Similarly, the template used in this assay is N and E plasmid mix with 1 to 1 ratio. Our result showed that 10⁷ to 10¹ copies turned yellow color, and the non-template control remained pink (see FIG. 10, C). In addition, 10⁷ copies reaction incubating at 4° C. remained pink color, which suggested the color changes in these assays were due to LAMP amplification but not nucleic acid adding into reagents (see FIG. 10, D). In conclusion, we choose N and E primer set 1 mix for following tests.

### 3. Performance of TANbead Automated Extraction and Assay System.

First, we evaluated the elution efficiency between two elution steps by qPCR assay. It is important to control the two elution steps releases same amount of isolated RNA so the detection results of the two eluents (EB1 and EB2) can be similar. Therefore, the elution time of EB1 and EB2 are different. Similarly, the plasmid controls of E gene from SARS-CoV-2 were ten-fold serially diluted in PBS buffer and extracted by Maelstrom™ 8 Autostage, then eluted into two elution buffers respectively. Next, the E gene plasmid yields in EB1 and EB2 were detected by qPCR to compare two elution steps result respectively.

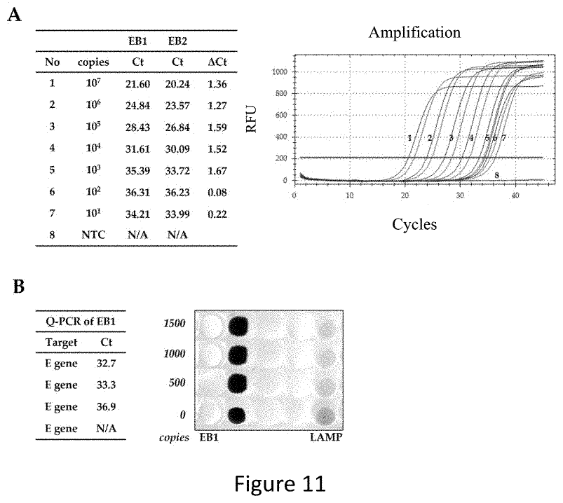

In A of FIG. 11, determination of the elution efficiency by qPCR. The Ct values of E gene plasmid control in EB1 and EB2 were showed in left panel and the ΔCt was obtained by subtracting Ct of EB1 from Ct of EB2. The amplification plot of E gene plasmid control in EB1 and EB2 was showed in right panel. No. 1˜7 was ten-fold dilution of template. No. 8 was a non-template control. In B of FIG. 11, it shows the results of TANBead automatic extraction and assay system. Pseudovirus spike-in nasopharyngeal swab samples were extracted and detected by qPCR for EB1 and RT-LAMP for EB2 respectively. The Ct values of EB1 were showed in left table and the colorimetric RT-LAMP results were showed in right panel.

As shown in A of FIG. 11, overall, the difference between the Ct values of EB1 and EB2 were less than 5%. Notably, the Ct of EB2 is generally lower than EB1 due to longer elution time. This is designed to make the results of the first screening by RT-LAMP more reliable and faster, since the EB2 is for RT-LAMP. However, we noticed that in low copies (10² to 10¹) plasmid extraction, EB1 and EB1 exhibited very close Ct value. It may be due to the limit of extraction. Nevertheless, we believe that the small difference of Ct value between EB1 and EB2 does not affect differential diagnosis.

To test the performance of this system, we used various copy number of pseudovirus spike-in nasopharyngeal swab as extraction sample. In addition, we replaced the EB2 with RT-LAMP reagent, allowing to perform the detection of viral RNA after extraction procedure. Moreover, the EB1 were analyzed by qPCR to provide the quantitative Ct value. As shown in FIG. 11B, 1500, 1000, and 500 copies were turned yellow color indicating positive result, and the negative control remained pink color as expected. Furthermore, the qPCR results were consistent with RT-LAMP, the E gene was detected in EB1 with pseudovirus input. In conclusion, our data indicated that this system is able to perform extraction and detection of viral RNA.

### 4. Cross-Contamination Test of TANbead Automated Extraction and Detection System

Although automated extraction system brings convenient and fast detection system, the risk of cross contamination during extraction steps between neighboring wells should be considered. In addition, RT-LAMP is highly sensitive thus may be easily contaminated and resulted in false positive. Therefore, we tested whether the cross-contamination to neighbor well exist during TANBead automated extraction and detection. The samples were pseudovirus spike-in nasopharyngeal swab as previous assay, the positive and negative samples were arranged to neighbor well and performed extraction and detection procedure.

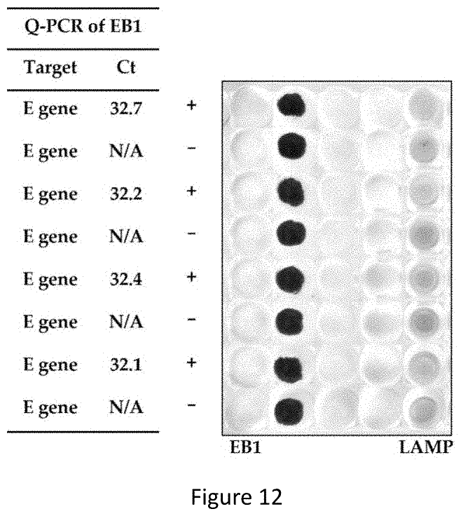

FIG. 12 illustrates cross-contamination test of TANBead automatic extraction and detection system. Nasopharyngeal swab with and without spike-in pseudovirus (1500 copies) were extracted in neighboring well and detected by qPCR and RT-LAMP respectively. The Ct values of E gene in EB1 were showed in left panel; colorimetric RT-LAMP results were showed in right panel.

As shown in FIG. 12, the negative controls were remained pink in EB2, and the qPCR showed non-detected of E gene. This data indicated that cross-contamination to neighbor well was not observed in auto plate during extraction procedure. The contamination test was repeated using different lot numbers of auto plate and showed consistent results, which suggesting the assay is stable and reproducible.

In conclusion, one embodiment of the present disclosure, we developed an automated extraction and detection system, which contains double elution steps to provide visual RT-LAMP results and ready-to-use samples of RT-qPCR. This system not only reduces hands-on time and time-to-results but also increases the throughput of diagnosis, it may be a useful method during epidemic prevention. However, this system has potential to expand for further applications. Dengue fever, influenza, and Zika virus infection are all important diagnostic targets of infectious diseases. Moreover, based on this system, it is possible to create a high-throughput genotyping system with special designed primers, such as Dengue virus typing, SARS-CoV-2 variants identification, SNP gen-253 otyping, etc.

According to the technical feature described above, the automated system disclosed in the present disclosure is designed for mid-to-high throughput nucleic acid extraction application. Specialized spin tips bring in high efficiency in mixing samples, the isolation principle is the collection and transfer of magnetic beads which adsorbs nucleic acid from well to well, and purified DNA and RNA can be obtained after binding, wash, and elution. As such, through using the system for automatic nucleic acid extraction and qualitative analysis disclosed in the present disclosure, user may save more time and labor to obtain a high efficiency and high accuracy nucleic acid extraction and analysis application.

Although the present invention has been described in terms of specific exemplary embodiments and examples, it can be appreciated by those skilled in the art that changes could be made to the examples described above without departing from the broad inventive concept thereof. It is understood, therefore, that this invention is not limited to the particular examples disclosed, but it is intended to cover modifications within the spirit and scope of the present invention as defined by the appended claims.

It is to be understood that both the foregoing general description and the following detailed description are exemplary and explanatory only and are not restrictive of the invention, as claimed.

What is claimed is:

1. A system for automatic nucleic acid extraction and qualitative analysis, comprising:
a magnetic rotary mixer, comprises:
a plurality of magnetic rods for generating magnetism, configured to be retractable from the magnetic rotary mixer;
a plurality of spin shaft for mounting tips, and the plurality of magnetic rods extend therein;
an auto stage, comprises:
a plate holder, which allows a plate place thereon;
a mixer holder to hold the magnetic rotary mixer over the plate holder; and
a heat plate, disposed under the plate holder for heating the plate.

2. The system of claim 1, wherein the plate holder is horizontally movable.

3. The system of claim 2, wherein the plate holder is moved by a stepper motor.

4. The system of claim 1, wherein the mixer holder is vertically movable.

5. The system of claim 4, wherein the mixer holder is moved by a stepper motor.

6. The system of claim 1, wherein the magnetic rotary mixer comprises 8 spin shafts.

7. The system of claim 1, wherein the magnetic rotary mixer further comprises a control panel for controlling a condition of the nucleic acid extraction.

8. The system of claim 1, wherein the plate has 96 wells.

9. The system of claim 1, which further comprises a cover shell.

10. The system of claim 1, wherein the spin shaft is rotated by a motor.

11. The system of claim 1, wherein the auto stage comprises a controlled chip with preset programs.

12. A method for automatic nucleic acid extraction and analysis performed by the system of claims 1, comprising:
introducing samples, reagents and beads into the plate;
conducting a nucleic acid extracting step, the magnetic rotary mixer mixes the samples, the reagents and the beads, and extracts the nucleic acid thereof with the beads; and
conducting an analysis step by RT-LAMP,
wherein the plate and the magnetic rotary mixer are moved automatically when conducting the nucleic acid extracting step.

13. The method of claim 12, wherein the plate and the magnetic rotary mixer are moved by a stepper motor.

14. The method of claim 12, wherein the plate and the magnetic rotary mixer are moved horizontally and vertically respectively.

15. The method of claim 12, which further comprises a heating step for controlling the temperature of assay step.

16. The method of claim 15, wherein the heating step is performed by the heat plate.

17. The method of claim 12, wherein a reagent of RT-LAMP comprises primers that can combine with the nucleic acid and moderate pH.

18. The method of claim 17, wherein the reagent of RT-LAMP further comprises pH indicator.

19. The method of claim 12, wherein the beads are magnetic beads.
